(19) United States
(12) Patent Application Publication
(10) Pub. No.: US 2025/0366930 A1
(43) Pub. Date: Dec. 4, 2025

(54) SYSTEM AND METHOD FOR PLACEMENT OF NEUROSTIMULATION LEADS USING AUGMENTED REALITY

(71) Applicant: Axonics, Inc., Irvine, CA (US)

(72) Inventors: Guangqiang JIANG, Irvine, CA (US); Yuqing WAN, Irvine, CA (US)

(73) Assignee: Axonics, Inc., Irvine, CA (US)

(21) Appl. No.: 19/221,396

(22) Filed: May 28, 2025

Related U.S. Application Data
(60) Provisional application No. 63/654,618, filed on May 31, 2024.

(51) Int. Cl. A61B 34/20; A61B 90/00

(52) U.S. Cl. CPC A61B 34/20; A61B 90/37; A61B 2090/363; A61B 2090/365; A61B 2090/3966

(57) ABSTRACT

A system and method for locating and guiding a peripheral nerve evaluation (PNE) lead. The system comprising an X-ray system to capture images of the patient. The images are processed by the system and are provided to a user to utilize an augmented reality (AR) system for inserting a needle for placing leads for the PNE procedure.

### CROSS-REFERENCE TO RELATED APPLICATION

This application claims priority to and the benefit of U.S. Provisional Patent Application No. 63/654,618 (filed May 31, 2024) which is incorporated by reference herein in its entirety.

### GENERAL DESCRIPTION

This application relates to devices and methods to assist with the placement of leads used in neurostimulation. In the exemplary embodiment, the device and method relate to the placement of an electrical lead used in sacral neuromodulation, and more particularly, to a system and method for locating the sacral foramina during a peripheral nerve evaluation (PNE) procedure in order to place the electrical leads of a PNE system in the appropriate position.

Sacral neuromodulation is a treatment for bladder and bowel dysfunction, which involves implanting a device that provides controlled electrical stimulation to the sacral S3 spinal nerve in the patient. Prior to receiving a permanent implant, the patient undergoes a procedure called a peripheral nerve evaluation (PNE). The procedure involves implanting temporary leads into the patient with the leads connected to an external pulse generator, and then observing results for a period of time, usually anywhere between 3 to 14 days. If the results meet certain clinical standards, then the patient may be a candidate for receiving an implantable pulse generator for sacral neuromodulation.

Current techniques for lead placement during the evaluation procedure involve identifying palpable skeletal or bony landmarks on the patient and inserting a foramen needle into the patient based on the location of these landmarks. The objective during this procedure is to insert the foramen needle through the skin and into the S3 foramen such that an electrical stimulation lead may be provided along the sacral S3 spinal nerve. The procedure may be performed in an operating room setting with fluoroscopic or other image guidance to provide for more accurate placement of the leads. However, this procedure is also often performed in an office setting under local anesthesia and without imaging. In an office setting, the placement method is essentially a “blind” insertion method because placement is not informed by a picture or fluoroscopy of the patient's anatomy.

When fluoroscopy or other imaging is not used, such as in the office setting, the physician inserts the foramen needle from outside the patient's body and into the S3 foramen based on experience and by referencing the palpable landmarks. The physician cannot see the S3 foramen when they are attempting to place the foramen needle through the S3 foramen. The use of palpable skeletal or bony landmarks is based on normal anatomy without consideration for anatomic or pathologic variations. This may lead to improper placement of leads in an office setting and eventual failure of the PNE. For example, often plural attempts are required to locate the S3 foramen and successfully insert the foramen needle through it. In some instances, due to multiple failed attempts at proper needle placement, a patient may even abandon the PNE (and thus sacral neuromodulation altogether) without ever having the leads properly placed. Misplaced leads may also lead to a less than ideal clinical outcome and a premature abandonment of an otherwise efficacious treatment. Thus, there remains a demand and need to provide an improved system and method for PNE lead placement.

### SUMMARY

The embodiments disclosed herein relate to devices and methods that improve efficacy and efficiency of locating the sacral foramina during a sacral neuromodulation procedure. Embodiments include imaging a portion of the patient, identifying internal points of the patient in the imaging, calculating measurements based on the imaging and the identified points, transferring the measurements to a head mounted display. The head mounted display can project the measurements directly onto a display, providing a clear visual reference. The electronic device may also be capable of capturing real-time video or images of the patient, allowing for the calculated measurements to be projected or superimposed directly onto the live video feed or captured image, offering real-time guidance during the procedure. The disclosed embodiments help the physician more accurately locate the S3 foramen and provide an improvement over conventional techniques that are less accurate at locating the S3 foramen during a blind insertion. In one embodiment, the electronic device may also be an augmented reality device capable of having the calculated measurement superimposed directly onto the glass such that the measurements are seen on the real-world object viewed through the glass.

In one embodiment, a separate display can also be utilized in tandem or as a substitute to the head mounted display. This display can aid the physician in a similar way by displaying the required points. Additional external sensors may support the head mounted display and/or the separate display to aid in the locating of the sacral foramina.

An exemplary method for marking a location on a patient for inserting a needle for placement of a lead for nerve stimulation therapy is disclosed herein. The method includes obtaining a scaled image of a patient. The tip of a patient's coccyx shown in the image is identified and a first line is imposed on the image. The first line extends from the tip of the coccyx to a surface of the patient's skin. A second line is used to determine the location of a needle insertion, wherein the second line extends from a location in a sacral foramen in a direction perpendicular to the centerline of the sacrum to the surface of the patient's skin. The method includes calculating measurements corresponding to the target location on the patient. The measurements are transferred to a head mounted display (HMD), where the HMD provides an overlay and/or indicators that identify the target location on the patient.

### BRIEF DESCRIPTION OF THE DRAWINGS

FIG. 1 shows an exemplary partial view of the human anatomy.

FIG. 2 shows the elements of an exemplary medical element locating and placement system.

FIG. 3 shows an example of a radiopaque landmark device.

FIG. 4 shows an example of a radiopaque landmark device.

FIG. 5 shows a flowchart of an exemplary method of locating an insertion point and placement of a medical element.

FIGS. 6 and 7 show exemplary implementations of identifying points in a fluoroscopic image and processing the image for measurements.

FIG. 8 show an exemplary augmented reality system implemented for lead placement.

FIG. 9 show an exemplary augmented reality system implemented for lead placement.

FIG. 10 show an exemplary lead placement system.

FIG. 11 shows an exemplary head mounted display.

### DETAILED DESCRIPTION

The particulars shown herein are by way of example and for purposes of illustrative discussion of the disclosed embodiments and are presented to provide a readily understood description of the principles and conceptual aspects. In this regard, no attempt is made to show structural details in more detail than is necessary for a fundamental understanding, and the description taken together with the drawings make apparent to those skilled in the art how the disclosed devices and methods may be embodied in practice.

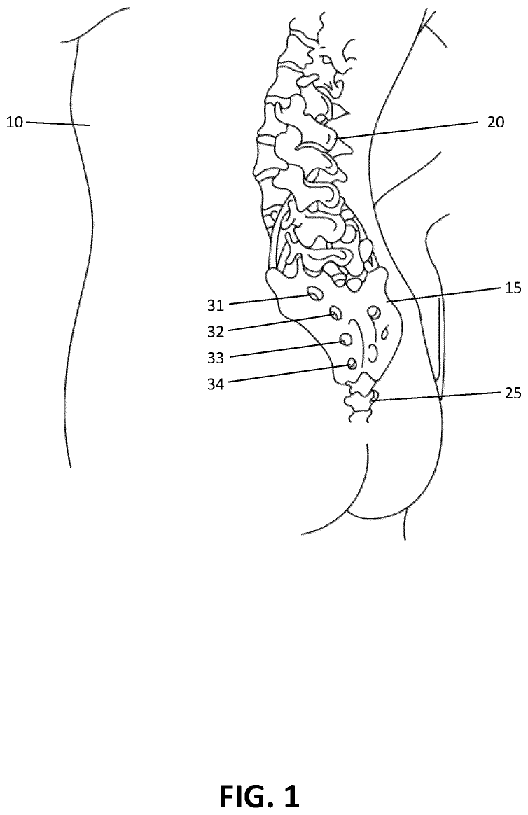

FIG. 1 shows an exemplary partial view of certain skeletal components of a human torso 10 including the sacrum 15 at the base of the spine 20, and the coccyx 25 at the base of the sacrum 15. As shown in FIG. 1, the sacrum 15 includes two groups of sacral foramina, or openings through which the sacral nerves pass, arranged in two vertical rows, each respectively located on either side of the medial sacral crest. In FIG. 1, foramen 31 corresponds to the S1 foramen, foramen 32 corresponds to the S2 foramen, foramen 33 corresponds to the S3 foramen, and foramen 34 corresponds to the S4 foramen. Only one group of sacral foramina is shown and numbered in FIG. 1. A system and method for accurately locating a selected one of the foramina 31-34 and inserting a needle through the selected one of the foramina to access a nerve for implanting a component of a sacral neuromodulation system is disclosed herein. Embodiments are described herein with respect to locating and inserting a foramen needle through the S3 foramen; however, the described system and method is not limited to use with the S3 foramen.

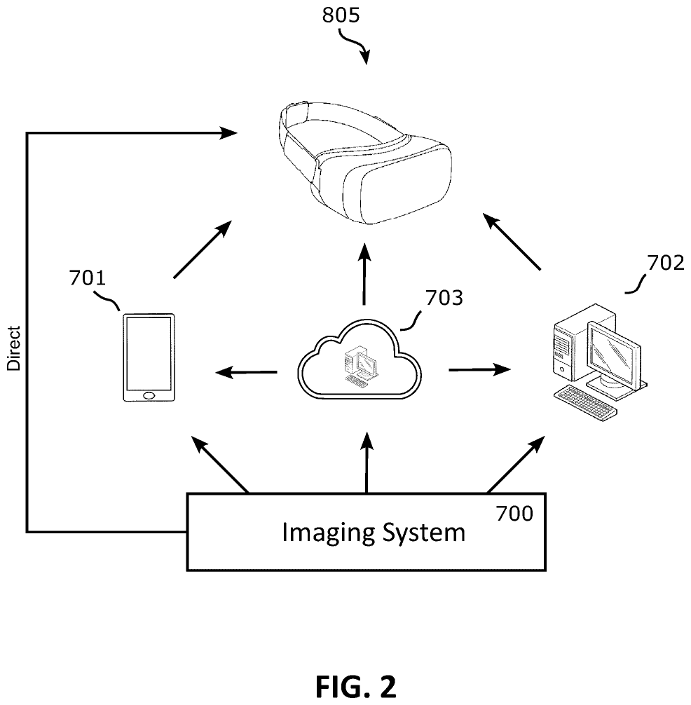

FIG. 2. shows the components of medical element placement system 100 which includes an imaging system 700 that may communicate with one or more electronic devices. Although X-ray or fluoroscopic imaging is preferred, other imaging technologies that show and distinguish internal anatomic structure(s) of the patient may be used. The electronic device may include a smartphone or other mobile device 701, a computer 702, or any other known electronic device that is capable of receiving inputs from the imaging system 700 and displaying outputs on the display of the electronic device. In addition to (or as an alternative to) the display, the electronic device may provide various indications to the user through audible or visual alerts or messaging. The imaging system 700 is configured to capture images of a patient requiring a PNE procedure. The medical element placement system 100 requires images to be taken in both the anterior-posterior (or posterior-anterior) and lateral planes in order to provide the measurements required to place the PNE lead in the correct location. Once the images have been captured, images from the imaging system 700 may be sent to the electronic device(s) which is configured to run or function with an application (e.g., software) that is configured to process the images and provide information that may be used to properly position a head mounted display 805 on the patient and mark the patient as necessary to identify the insertion point for a foramen needle used for placement of the PNE lead. In another embodiment, the outputs of imaging system 700 may be directly sent to the head mounted display (HMD) 805. The head mounted display may perform the same functions and include the same capabilities as the mobile device 701 or computer 702 as described.

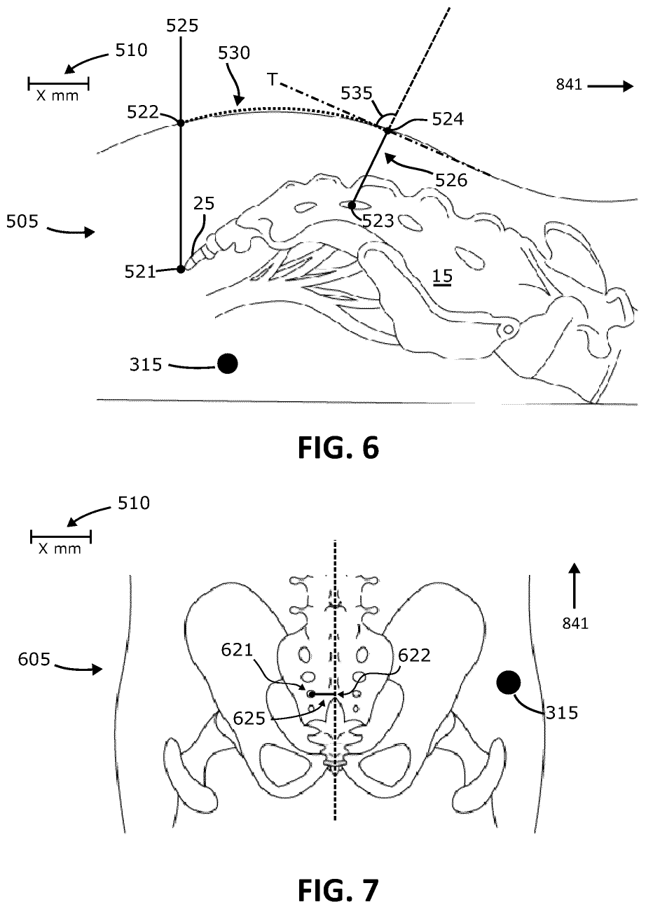

In one embodiment, the application may run using a cloud-based computing service 703 that communicates with the electronic device. The cloud-based computing service may perform the calculations required to identify a preferred point of needle entry (and angle) and provide the resulting location and angle to the electronic device. The electronic device may run the application (or a web client that accesses a cloud-run version of the application) described below with respect to FIGS. 6 and 7, and the application may provide various information on the display (e.g., screen) on the screen of the electronic device. For example, the electronic device may display the coordinates and angle to be used for needle insertion on the patient. The information (e.g. coordinates and angle) may be provided by the application can then be uploaded to the HMD 805 to aid in the placement and insertion of a needle onto the patient.

The program may also incorporate the use of machine learning or artificial intelligence (AI) in order to provide enhanced image recognition capabilities. The image recognition capability or feature may allow the program or application to automatically provide the points of interest, draw or impose the required lines on the image, and calculate measurements based on the images provided to the program. The image recognition feature may also provide the points of interest regardless of the orientation of the patient during the imaging. Thus, if the patient appears in an inconsistent position with regard to the axis of the image, the measurements may still be obtained due to the ability of the employed AI to recognize the anatomical points of interest. As mentioned above, the “drawing” of lines may be visible to the user of the program or merely performed inherently as part of the operation of the program or application that determines or calculates the required measurements and the preferred location for needle insertion.

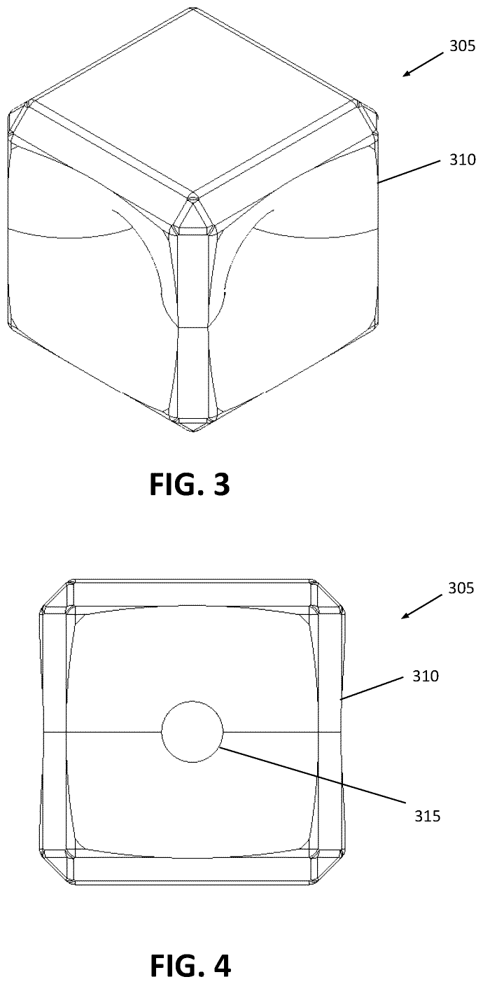

FIGS. 3 and 4 show an example of a radiopaque landmark device 305 that may be used with the imaging of the patient. The imaging includes an X-ray of the lateral plane of the patient showing both the sacrum 15 and coccyx 25 of the patient. The imaging may also include one or both of the anterior-posterior (AP) or posterior-anterior (PA) planes of the same area (i.e., showing the sacrum and coccyx of the patient). The radiopaque landmark device 305 may be placed on the patient during the X-ray imaging in the field of view of the imaging device 700. The radiopaque landmark device 305 includes a radio-transparent housing 310 and a radiopaque element 315. The radio-transparent housing 310 is composed of a material that is relatively transparent to an X-ray device (or whatever imaging technology is used). Plastic may be used for the radio-transparent housing 310, for example. The radio-transparent housing 310 provides for easy placement handling of the radiopaque landmark device 305 onto the patient when images are being taken. The radiopaque element 315 is composed of a material that is relatively opaque to an X-ray (or whatever imaging technology is used). Stainless steel may be used for the radiopaque element 315, for example. The radiopaque element 315 has a predefined dimension that is used with the X-ray image(s) to define a scale of the image(s) of the patient and provide a reference to properly determine the measurements required to locate the insertion point for a needle on a patient. In a particular embodiment, the radiopaque element 315 includes a sphere of a predefined diameter so that the radiopaque landmark device 305 may be placed in any orientation on the patient during the imaging. Many other known landmark devices may be utilized, such as radiopaque devices with different predetermined shapes and dimensions. Landmark devices may be fixed onto the patient or the radiographic table where X-ray image is taken.

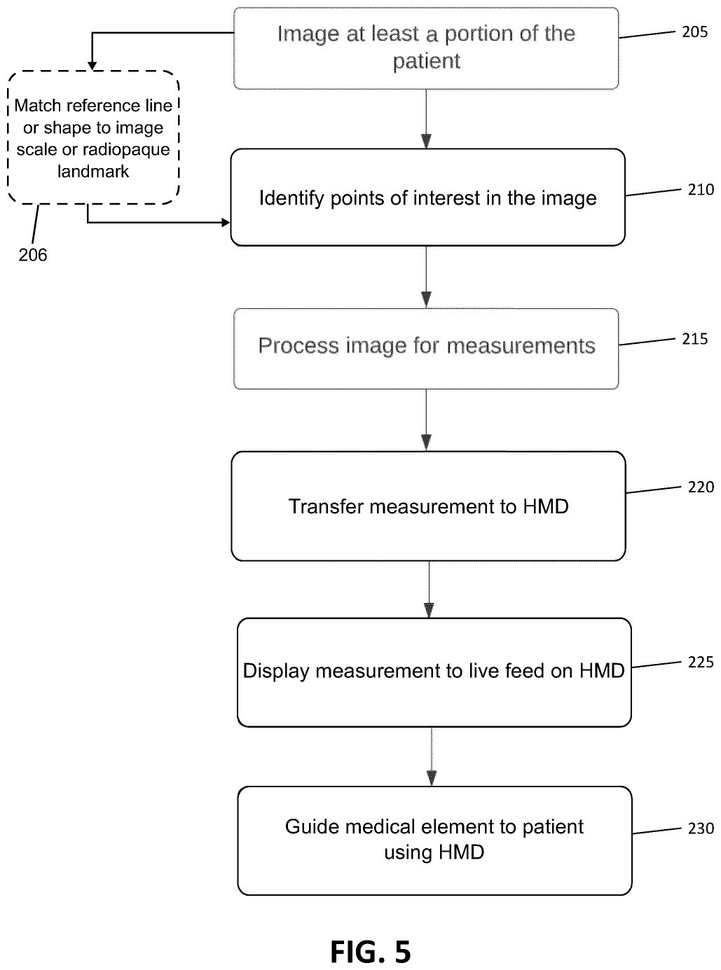

FIG. 5 shows a flowchart of an exemplary method. Step 205 includes imaging a portion of the patient. The images of the patient may be obtained with the patient in various positions. The imaging system 700 may capture images of the patient's sacrum 15 and coccyx 25 in both the anterior-posterior (or posterior anterior) and the lateral planes. The processing software of the imaging system 700 may set the appropriate dimensions using a scale provided by a radiopaque landmark device or by the imaging system 700. The processing software of the imaging system 700 may automatically manipulate the image further in order to clarify the image (e.g. modifying contrast or brightness of the image). The image may also be further clarified once transferred to the electronic device.

Step 210 includes identifying points of interest related to the patient in the imaging to determine key measurements used in the calculation. Step 215 includes calculating measurements that identify the recommended insertion location of the needle based on the imaging and the identified points. These measurements may be used to display the insertion location on the HMD 805. The software may also calculate the needle entry angle α and the minimum needle length. The minimum needle length is defined as the minimum needle length that is required to reach the target location (e.g., the S3 sacral foramen).

Step 220 includes transferring the measurements calculated in Step 215 to the HMD 805. Step 225 includes using HMD 805, where the HMD will display the calculated target point on a live image of the patient being viewed through the display. Step 225 superimposes the measurements onto the image of the patient within the live feed. This creates an augmented reality (AR) overlay that assists in precise needle placement at step 230. In step 230, the user utilizing the HMD will guide a medical element (e.g., a foramen needle) into the patient. Because the HMD superimposes the measurements onto the live video images, the user can clearly visualize the target point while inserting the needle. Embodiments implementing these steps will become apparent from the following figures and associated descriptions.

FIGS. 6 and 7 show an exemplary X-ray image 505 in which the radiopaque element 315 of the radiopaque landmark device 305 is visible. Because the radiopaque element 315 has a predefined diameter, this known measurement of the radiopaque element 315 may be used to define a scale for the X-ray image 505. This scale may be used by the application when calculating measurements as described herein. A scale indicator 510 (e.g., a scale line embedded and depicted on an x-ray image) may also be provided as an alternative to the landmark device 315, when the imaging system 700 is capable of including an automatic scaling function that provides a scale of the image. The scale indicator 510 may adjust to correspond to the selected image magnification by a user. In both alternatives (e.g., using the radiopaque landmark device 305 or the automatic scaling function), the image is provided with a scale of the image that may be used when calculating the measurements necessary to identify the location for needle insertion required for proper lead placement. When an image scale 510 cannot be provided by the imaging system 700, the radiopaque landmark may be used so that the image can still be properly measured.

FIGS. 6 and 7 also show exemplary implementations of identifying points in the image(s) and processing the image(s) for measurements that may be used at steps 210 and 215, respectively. The measurements refer to the coordinates or location of the needle insertion point on the patient. As mentioned above, these measurements can be transferred to the HMD 805. The image(s) from step 205 are uploaded to an electronic device (not shown) for processing. The electronic device may be, for example, a desktop computer, laptop computer, tablet computer, or a smartphone that runs a specialized software (e.g., a proprietary application). The application may be part of a software program product as described herein. Alternatively, all or part of the processing may be conducted in a cloud-based application.

The electronic device may include a display for displaying images (e.g., X-rays taken at step 205) and a user input mechanism that permits the user to identify points in a displayed image. The display and user input mechanism may be combined in a touchscreen display, for example, that can display an image and receive user touch input defining points of interest in the image. The display and user input mechanism may be separate, for example, such as a display screen that displays an image and a mouse or trackball that controls a pointer (e.g., cursor, arrow, etc.) superimposed on the displayed image, and a button the user may depress to provide define a current location of the pointer on the image as a point of interest in the image.

The step of identifying points in the image in step 210 includes importing the one or more images from step 205 into the application running on the electronic device. The application establishes a scale of the image(s) using either the predefined dimension of the radiopaque element 315 in the image(s) or a scale indicator (e.g., such as scale indicator 510) provided with the image(s). Step 210 may optionally include the application adjusting visual aspects of the image(s), such as contrast, etc. FIG. 6 shows an example of a simplified lateral X-ray image 505 that includes a scale indicator 510 that defines a scale of the image 505.

With continued reference to FIG. 6, the application receives user input via the electronic device. The user uses the image to identify the points of interest in the image 505. The user may interact with the image (e.g., via a touch screen, keyboard, stencil, etc.) to identify the points of image. There are four points of interest 521, 522, 523, and 524 that are defined by user input. The application may provide one or more messages to the user that prompt the user to provide their input for one or more of the points of interest. The user provides input to define the first point of interest 521 at the tip (e.g., distal end) of the coccyx in the image 505. In response to receiving this input, the application draws a vertical line 525 upward from the first point of interest 521. While the line 525 is displayed, the user provides input to define the second point of interest 522 at the intersection of the line 525 and the outer surface of the skin of the patient. The user provides input to define the third point of interest 523 at the center of the targeted foramen, in this case the S3 sacral foramen, in the image 505. In response to receiving this input, the application draws a line 526 perpendicular to the sacrum at third point of interest 523 and upward toward the outer surface of the skin of the patient. While the line 526 is displayed, the user provides input to adjust or confirm that the line 526 is perpendicular to the sacrum and provides input to define the fourth point of interest 524 at the intersection of the line 526 and the outer surface of the skin of the patient.

In the event that the images are not fully legible (i.e., S3 not fully visible) the application may suggest where to target the S3 foramen based on other points of interest. For example, as an alternative to asking for point of interest 523, the program may ask locations of the lumbosacral joint (the junction of L5 vertebrae and S1) and the caudal tip of the coccyx 521. The program may then estimate the location of 523 by calculating a point halfway between the lumbosacral joint and the caudal tip of the coccyx 521. Other alternatives may include providing estimations of where the S3 foramen is located by providing known measurements of the average location of the S3 foramen of the human anatomy (e.g., S3 is approximately 11 cm from the tip of the coccyx).

The method described herein may include utilizing image processing software. For example, the application interacting with the electronic device may include image processing software to facilitate processing the image. The image processing software may provide the user with the ability to utilize the following functions with regard to the image: pan, zoom, windowing, scroll, crosshair, filtering (brightness and contrast adjustment), measurement of distance, angles and areas, image rotation/flip, etc. The aforementioned image processing capabilities may run in the background of the application and software without user input, or may be provided as options for the user to manipulate the image in order to assist with the identification of points of interest.

The image processing software may be specialized for handling the image files typically associated with fluoroscopy images (e.g., DICOM files). Digital Imaging and Communications in Medicine (DICOM) is the international standard for medical images and related information. The standard defines the formats for medical images that can be exchanged with the data and quality necessary for clinical use. The processing software may provide the ability to create a 3D reconstruction model of the patient and the patient's skeletal structure in order to assist in the location of the sacral foramen and preferred needle entry point. In addition, the image processing software may be configured to anonymize and de-identify any patient details retained in the image so that the image can be employed in machine learning and AI applications.

Processing the image for measurements at step 215 includes determining a length measurement, an angle measurement, and a depth measurement based on the points of interest defined by the user at step 210. The application determines the length measurement, the angle measurement, and the depth measurement based on: the coordinates (e.g., X-Y cartesian coordinates) of each of the points of interest in a coordinate system defined for the image; the scale of the image relative to the same coordinate system; and one or more predefined dimensions of the patient's body. The one or more predefined dimensions of the patient's body include a segment length of the patient's body calculated from the images taken of the patient. The application uses this information (e.g., the coordinates, the scale, and the predefined dimensions of the patient's body) as inputs to an algorithm or program that employs established geometric and trigonometric formulas and calculations to determine: (i) a length of a segment 530 that extends between the second point 522 and the fourth point 524 where the segment follows the surface of the patient's body along the area of interest; (ii) an angle 535 between the line 526 and a tangent of the segment 530 at the fourth point 524; and (iii) a length of the line 526 between the third point 523 and the fourth point 524. The determined length of the segment 530 between the second point 522 and the fourth point 524 includes the length measurement, the determined angle 535 includes the angle measurement, and the determined length of the line 526 between the third point 523 and the fourth point 524 includes the depth measurement. The application outputs the determined measurements to the user, e.g., via display. The angle 535 may be calculated as the angle between an extension of the line 526 and a tangent line “T” that is tangent to the segment 530 at point 524.

FIG. 7 shows an example of a posterior-anterior (PA) X-ray image 605. The application receives user input via the computing device when the user identifies points of interest in the image 605. The user may interact with the image (e.g., via a touch screen, keyboard, stencil, etc.) to identify the supplemental points of interest 621, 622 on the image. The first supplemental point of interest 621 is located at the center of the S3 foramen in the image 505. In response to receiving this input from the user, the application draws a line 625 that intersects the point 621 and is perpendicular to the centerline of the sacrum. While the line 625 is displayed, the user identifies the second supplementary point of interest 622 at the intersection of the line 625 and the centerline of the sacrum. In this example, the application uses the coordinates of the points 621 and 622 to determine a lateral distance between the points 621 and 622. The application outputs the determined measurement to the user, e.g., via a display. The user may edit any one or more of the points of interest (i.e., 521, 522, 523, 524, 621, 622) and the program may automatically recalculate the measurements outputted based on the updated point(s) automatically. The step of identifying the supplementary points of interest may be omitted, and the application can assume a standard distance in the range of about 20 to 25 mm, based on published research or studies.

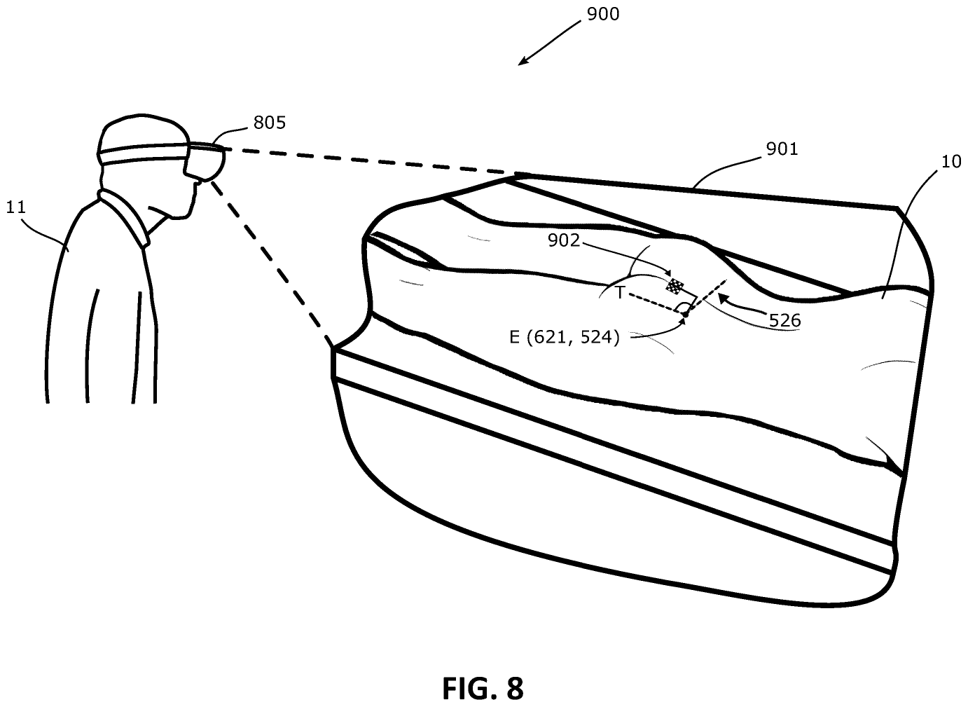

FIG. 8 illustrates an exemplary augmented reality system 900 employed in step 225. A user 11, such as a medical professional, utilizes the HMD 805 to view a live video feed 901 of the patient 10 in a prone position. The patient 10 may be provided with a fiducial marker 902 placed on their skin at a location corresponding to a vertical line extending from the tip of the coccyx (e.g., point 522 in FIG. 6). A fiducial marker is a small object placed either inside the body or on an external object that serves as a fixed reference point in various imaging and measurement systems. Fiducial markers placed on objects in the real world allow computer vision systems to track the object's position and orientation. This fiducial marker also serves as a reference for the HMD 805 to accurately display the calculated measurements superimposed on the live video feed 901. Based on the fiducial marker's position, the coordinates of the entry point E are determined within the live feed. The fiducial marker provides the HMD with the necessary scale for dimensions, orientation, and angles to calculate an accurate measurement for the target entry point E. This target entry point E, calculated in the previous steps (i.e., the combined coordinates of points of interest 621 & 524), is then superimposed onto the patient's image within the live video feed displayed by the HMD 805.

In one embodiment, the physician may also utilize palpation in order to find the coccyx tip of the patient. The HMD may track the physician's fingers, and the tip of the coccyx may be confirmed on the patient's skin by confirming the location point (tip of the coccyx) in the HMD. This would define a control point or reference point like a fiducial marker. Then the segment 530 can be defined by tracking the physician's fingers along the patient's spine. Once the location of the tip of the coccyx (e.g. 521) and the segment 530 is set and defined by the HMD, the measurements for the point of interest (621, 524) can be applied as discussed above and as shown in FIGS. 6 and 7.

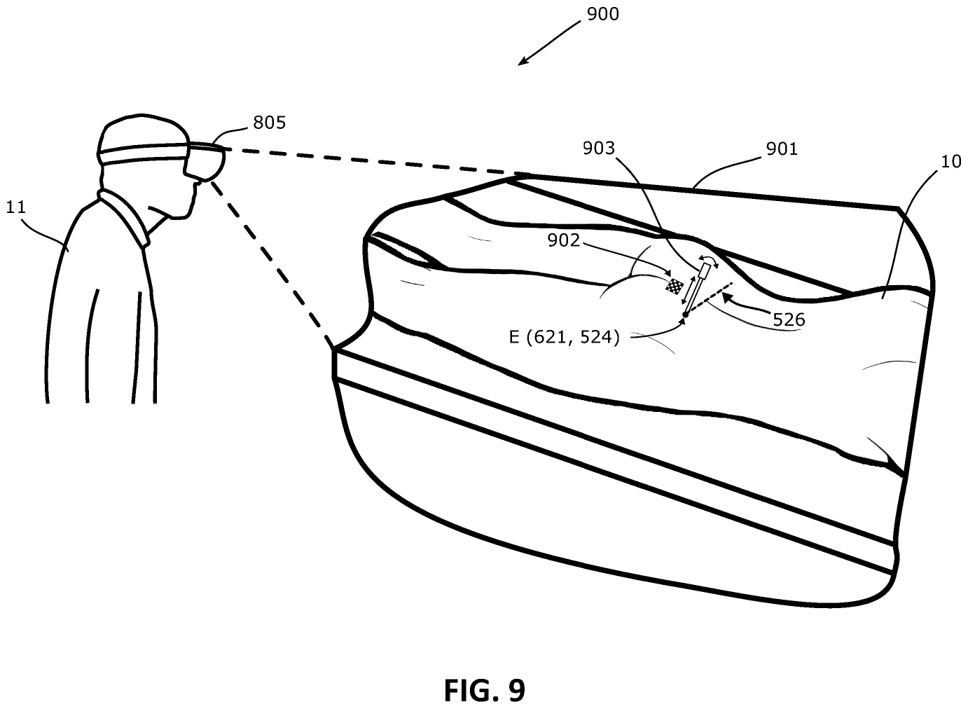

Referring to FIG. 9, the exemplary augmented reality system 900 is shown at step 230. In this step, the user 11 guides the medical element 903 towards the target entry point E. The user aligns the medical element's angle of insertion with the calculated angle of entry 535 (as described and shown in FIG. 6). The depth of insertion is determined by the calculated length of line 526 between the third point 523 and the fourth point 524 (as described and shown in FIG. 6).

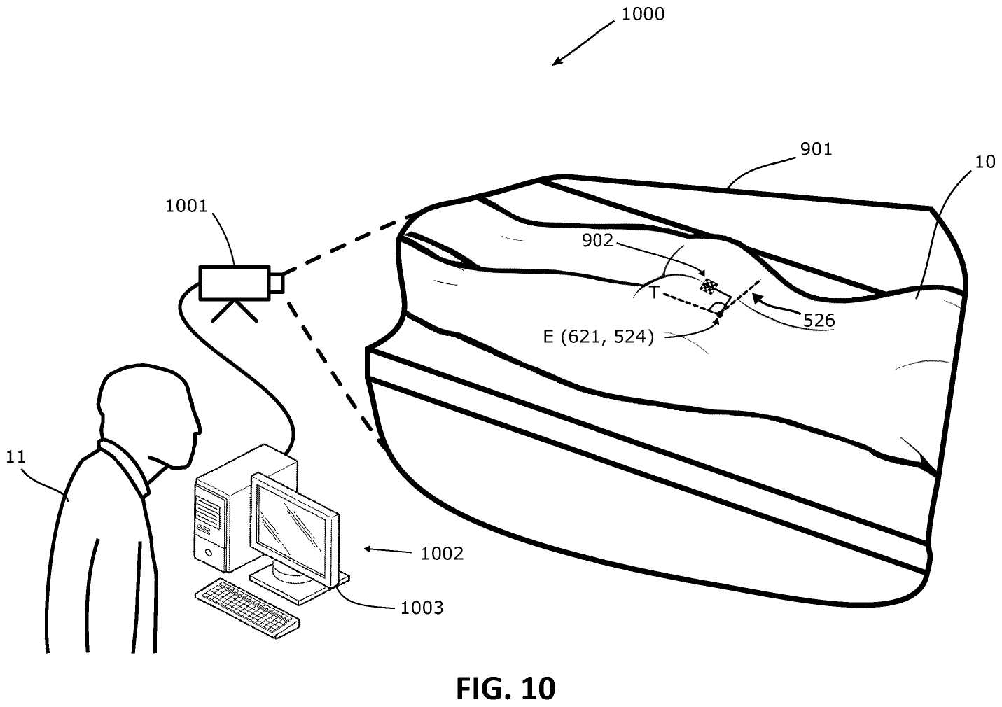

In one embodiment, the optical sensor, the display, and distance sensor may be located as separate units outside of an HMD. For example, a laptop or computer containing a display connected to separate optical sensor and distance sensor may be utilized instead of an HMD. The user 11 may instead utilize the laptop display in order to guide the medical element 903 to the correct angle and desired depth. As shown in FIG. 10, the embodiment of a foramen locating system 1000 is shown. As an alternative to the HMD 805, a computing device 1002 (e.g. laptop or computer) having a display 1003 may receive data from an optical sensor 1001. The user 11, may perform the same procedures as discussed above for the HMD. However, instead of utilizing an HMD, the user 11 may rely on the computing device 1002 instead and perform the procedure with the assistance of the display 1003. The separated optical sensor 1001 is configured to receive live video feed similar to the optical sensor found in the HMD. In one embodiment, the optical sensor 1001 may also directly project the measurements and calculated point of interest onto the patient's back, allowing the user to guide and insert the needle for lead placement.

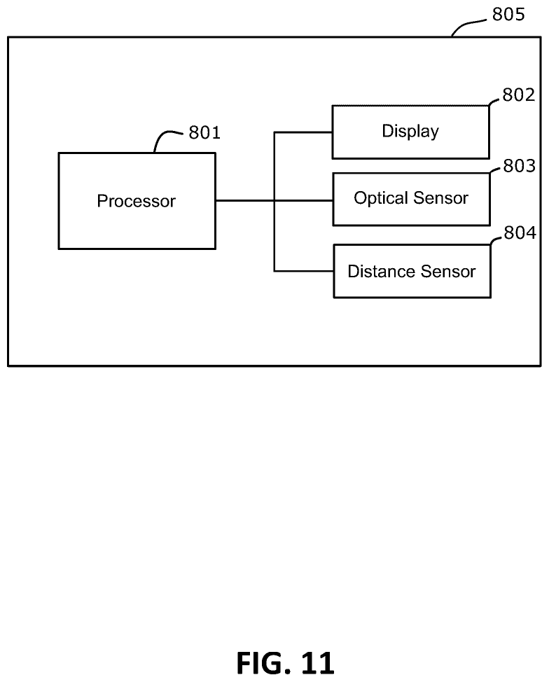

FIG. 11 illustrates the components of the exemplary HMD 805. The HMD 805 may include an optical sensor 803, a display 802 to receive inputs from the optical sensor, a distance sensor 804 in order to aid the processor 801 in calculating the distances, orientations, and angles needed to accurately superimpose the calculated target point E onto the patient's body in the live video feed. In one embodiment, the distance may be calculated through the optical sensor (e.g. via stereo vision) rather than a distance sensor.

In another embodiment, the described system may be used in applications in cardiac lead implantation. Traditionally, cardiologists depend on C-arm fluoroscopy to guide lead placement in real-time. However, to minimize radiation exposure for both patient and physician, it's desirable to reduce both the dosage and duration of fluoroscopic imaging. Preoperative 3D anatomical models of the patient's heart and relevant vasculature (axillary, subclavian, or cephalic veins) can be constructed from CT or MRI scans. During the implant procedure, this model is superimposed on the patient's body. The C-arm's real-time imaging, either through video streaming or cameras integrated with the HMD 805, provides visualization of the implanted lead and its electrodes. After the initial electrode is localized, the HMD 805 combines this data with the preoperative model to track and infer the electrode's location as the lead is advanced. This inferred positional information, visualized within the live feed 901, guides the physician during placement. Final confirmation of electrode placement with minimal C-arm imaging can then be performed, successfully reducing overall X-ray exposure.

In another embodiment, the described system may be used in applications in spinal cord stimulator (SCS) lead implantation. During SCS procedures, electrodes are placed within the epidural space, the region between the spinal cord and vertebrae. Preoperative imaging, such as CT scans or MRIs, provides valuable anatomical data about the spinal cord and surrounding structures. To ensure accurate alignment between the preoperative images and the live view from the AR device, a process called image co-registration is employed. This embodiment offers two primary methods for co-registration: skin markers, where fiducial markers are strategically placed on the patient's skin, or image feature matching, where the system leverages natural anatomical features for automatic alignment. The lead placement planning can be visualized in a similar way to other embodiments described above. The lead electrode location can be inferred by tracking the insertion length, in a similar way to embodiments described above. This solution minimizes C-arm usage.

In another embodiment, the described system may be used in applications in deep brain stimulator (DBS) lead implantation. Epileptic foci, the regions of the brain generating seizures, can be identified using preoperative techniques like electroencephalography (EEG) or functional magnetic resonance imaging (fMRI). Stimulating these foci with DBS electrodes disrupts seizure activity. Therefore, precise placement of electrodes at or near the identified foci is crucial. Preoperative 3D localization techniques can pinpoint the location of these foci. This critical information can then be visualized within the system 900 described herein. During open-skull DBS surgery, the neurologist can leverage this AR overlay with HMD 805 to visualize the brain anatomy and the precise location of the epileptic foci. This visual guidance facilitates optimal placement of the DBS electrodes.

In another embodiment, the described system may be used in applications in orthopedic surgery. The augmented reality system 900 as described herein offers significant benefits for orthopedic replacement surgery. During procedures, surgeons can view virtual models of replacement parts and the surgical plan directly overlaid onto the patient's body. This real-time, 3D visualization can enhance surgical precision.

In another embodiment, any pre-operative imaging information can be shown with AR technology for treatment planning and real time visualization of the virtual anatomy, in a similar way as described above with system 900.

As will be understood from the present disclosure an exemplary method is disclosed that includes the steps of: determining one or more measurements from at least one image of a sacrum of a patient; transferring the determined one or more target points to a head mounted display; superimpose the target point onto a live video feed using the head mounted display; and while using the head mounted display and superimposed target point, guide the insertion of a medical element into the patient.

In embodiments, the imaging data may be directly sent to the HMD for processing as shown in the method in FIG. 5. That is HMD 805 may include the capabilities of device 701 or 702. HMD 805 may calculate the points of interest (i.e., 521, 522, 523, 524, 621, 622) and determine the entry point E, angle of entry 535, and needle depth given the points of interest within the display and interface of the HMD 805.

In embodiments, the distance sensor may be a light detecting and ranging (LIDAR), infrared, and/or stereo vision sensor.

In embodiments, the HMD may include one or more known AR/VR headsets such as the Meta Quest, Microsoft Hololens, or Apple Vision Pro or other custom hardware specialized to display the measurements of interest of the patient to the user to aid in locating the target foramen.

In embodiments of the method, the guide device may be adjustable and applying the determined one or more measurements with the guide device may include adjusting the guide device based on the one or more measurements.

In embodiments of the method, the landmark includes the patient's coccyx.

In embodiments of the method, the landmark includes a locating element affixed to the patient.

In embodiments of the method, the at least one image includes an image of a pelvis of the patient in a lateral plane. In embodiments of the method, the at least one image includes an image of the pelvis of the patient in a posterior-anterior plane or an anterior posterior plane. In embodiments of the method, the at least one image includes an X-ray or a CT scan.

In embodiments of the method, the one or more measurements are determined based on user input defining points of interest in the at least one image. In embodiments of the method, the points of interest in the image include a location of a foramen in the sacrum. In embodiments of the method, the one or more measurements are determined based on a predefined dimension of the guide device.

In embodiments of the method, the guide device on the patient's backside defines a location and an angle of entry of the medical element into the patient's body.

In embodiments of the method, the medical element includes a needle.

In embodiments of the device, the landmark includes a locating element affixed to the patient; and the locating feature includes a portion of the device that connects to the locating element. In embodiments of the device, the locating element includes a radiopaque marker.

In embodiments of the device, the medical element includes a needle.

In embodiments of the device, the entry location and angle of the medical element into the patient are configured to cause the medical element to pass through a selected foramen in the patient's sacrum.

In embodiments of the device, the medical element guide defines plural different angles for the angle of insertion of the medical element into the patient. In embodiments of the device, the plural different angles include plural different predefined angles that are defined by plural grooves in the medical element guide. In embodiments of the device, the plural different angles are defined by plural rotational locations of the medical element guide relative to the head.

As will be understood from the present disclosure a computer program product may be provided that includes one or more computer readable storage media having program instructions collectively stored on the one or more computer readable storage media, where the program instructions are executable to: receive at least one image of a sacrum of a patient; display the image; receive user input defining points of intertest in the displayed image; determine one or more measurements for a medical element guide based on the points of intertest and a predefined dimension of the medical element guide; and output the determined one or more measurements to a user.

In embodiments of the computer program product the points of interest include: a first point at a tip of the patient's coccyx or other anatomical landmark; and a second point at a foramen in the patient's sacrum. In embodiments of the computer program product the points of interest further include: a third point at an intersection of a surface of the patient's skin and a first line extending from the first point; and a fourth point at an intersection of the surface of the patient's skin and a second line extending from the second point. The anatomical landmark may be the L5 vertebra, the coccyx or other anatomical features, such as any distinct bony structure, soft tissue marker, or anatomical region.

In embodiments of the computer program product the medical guide element is configured to define a location and angle of insertion of a medical element into the patient while the medical guide element is located on the patient's backside.

Note that target points or measurements shown may be exaggerated and not to scale to provide clarity.

Additional embodiments may include using the device 805 as described herein. Also, instructions for using the device 805 as described herein may be provided. The instructions may be provided in print and/or in video.

A training platform may be provided for the disclosed method and device. The training platform is a software platform for doctors, sales reps, or any other person that needs to learn or practice the invented technique/method. The software platform allows users to upload mock patients and go through the measurement process, e.g., at steps 210 and 215. The software may be configured to grade the user on the accuracy of their user inputs and offer suggestions and tip on how to improve user input. The platform may be used to train and certify users virtually. The platform administrator can deploy training modules and updates to train and update users on the best practices. The platform administrator can also collect data on user experience and interaction. The platform can provide educational animations for various processes and procedures. Future data collection may be employed in this platform of later processing and optimization.

A training model may also be provided. The training model is a physical model used to train doctors, sales reps, physician assistances, nurses, etc., using the methods and devices 805 and/or 1105. The training model allows users to practice placement of the sacral lead alone or in use with the virtual training platform. The training model includes the sacral bone structure as well as surrounding bone and tissue structure important to this procedure. The training model includes a soft tissue simulating structure where the opacity may be adjusted to control internal visualization allowing users to block or see within the model. The training model includes targets that may be contacted or “hit” to confirm proper placement of the leads. When a target is hit a signal may be produced to confirm the proper placement. Additionally, the model anatomy may be adjusted to different levels to practice on different anatomies. This may be achieved by changing bone placement to change the dimensions needed to place the stimulator. This may also be achieved with different physical models altogether to represent different case complexities and scenarios.

Additionally, an AI platform may be provided. The AI platform may be configured to collect X-ray, measurement, and lead placement data that is obtained from monitoring the software platform, the success rate of patients, etc., and to use this data to optimize the lead/needle placements. The data may be used to facilitate machine learning to automatically identify points of interest for needle placement and the resulting measurements used for employing a guide tool on a patient. This data may be used to predict and optimize placement of leads resulting in more efficient conversions from an external pulse generator (EPG) to an implantable pulse generator (IPG).

An application (e.g., software) as described herein may include computing code stored on a computer readable storage medium and executed by processing circuitry of a computing device (e.g., computing device 700) to perform the functions described herein. The computing code may include routines, programs, objects, components, logic, data structures, and so on that perform particular tasks or implement particular data types that the code uses to carry out the functions of embodiments described herein. The application may be stored on a portable device (e.g., a USB drive). In addition, the application may include processing capability for handling the image files (e.g., DICOM files). Alternatively, the application may be configured to operate cooperatively from image processing software.

The foregoing examples have been provided merely for the purpose of explanation and are in no way to be construed as limiting of implementations of the present invention. While aspects of the present invention have been described with reference to an exemplary embodiment, the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations disclosed herein are not intended to be limited to the particulars disclosed herein; rather, implementations of the present invention extend to all functionally equivalent structures, methods and uses, such as are within the scope of the appended claims.

What is claimed is:

1. A method for marking a location on a patient and inserting a needle for placement of a lead for nerve stimulation therapy, the method includes the steps of:
obtaining a scaled image of a patient;
selecting a patient's anatomical landmark shown in the image;
imposing a first line on the image, wherein the first line extends from the patient's anatomical landmark to a surface of the patient's skin;
imposing a second line on the image, wherein the second line extends from a target location located in a sacral foramen in a direction perpendicular to the sacrum on the surface of the patient's skin;
calculating measurements corresponding to the location on the patient;
retrieving the measurements; and
transferring the measurements to a display, where the display overlays indicators representing the measurements to a visual representation the patient inserting the needle for placement of the lead based on the measurements.

2. The method of claim 1, wherein the display is a head mounted display.

3. The method of claim 1, wherein the display is computing device having a display that receives data from an external optical sensor.

4. The method of claim 1, wherein the step of obtaining a scaled image comprises using a radiopaque landmark device of known dimension.

5. The method of claim 2, wherein the visual representation of the patient is a live video feed of the patient.

6. The method of claim 5, wherein the indicators are superimposed onto the live video feed, creating an augmented reality overlay.

7. The system of claim 1, wherein the anatomical landmark includes one of a tip of a patient's coccyx and an L5 vertebra.

8. A system for marking a location on a patient for guiding placement of a neurostimulation lead, the system comprising:
an imaging system configured to obtain at least one scaled image of a pelvic region of a patient, said image including a sacrum and an anatomical landmark of the patient;
an optical sensor configured to capture a live video feed of the patient;
a processing unit configured to:
receive the at least one scaled image
impose a first line on the image, wherein the first line extends from the anatomical landmark to a surface of the patient's skin
impose a second line on the image, wherein the second line extends from a target location located in a sacral foramen in a direction perpendicular to the patient's sacrum to the surface of the patient's skin
calculate measurements corresponding to the location on the patient based on the first and second lines;
retrieve and transfer the measurements and the live feed to a display; and
overlay indicators representing the measurements onto the live video feed on the display, creating an augmented reality representation of the location on the patient.

9. The system of claim 8, wherein the imaging system is an X-ray system.

10. The system of claim 8, wherein the processing unit is further configured to allow user input to select the target location located in the sacral foramen.

11. The system of claim 8, wherein the display is a head mounted display.

12. The system of claim 8, wherein the display is computing device having a display that receives data from an external optical sensor.

13. The system of claim 8, wherein the processor further allows the user input to select the anatomical landmark shown in the image.

14. The system of claim 8, wherein the anatomical landmark includes one of a tip of a patient's coccyx and an L5 vertebra.

15. A computer system for marking a location on a patient for guiding placement of a neurostimulation lead, the system comprising:
a computing device having a non-transitory computer-readable storage medium having program instructions stored therein, wherein the instructions include:
receiving a scaled image of a patient;
imposing a first line on the image, wherein the first line extends from an anatomical landmark of the patient to a surface of the patient's skin;
imposing a second line on the image, wherein the second line extends from a target location located in a sacral foramen in a direction perpendicular to the patient's sacrum to the surface of the patient's skin
calculating measurements corresponding to the location on the patient based on the first and second lines
retrieving the measurements; and
outputting the measurements to a display showing the location onto the back of the patient on the display.

16. The system of claim 15, wherein the processor further allows the user input to select the anatomical landmark shown in the image.

17. The system of claim 15, wherein the display is a head mounted display.

18. The system of claim 15, wherein the display is computing device having a display that receives data from an external optical sensor.

19. The system of claim 15, wherein the display shows the measurements as an augmented reality overlay on a live video feed of the patient.

20. The system of claim 15, wherein the anatomical landmark includes one of a tip of a patient's coccyx and an L5 vertebra.
